(19) United States
(12) Patent Application Publication
(10) Pub. No.: US 2007/0293752 A1
(43) Pub. Date: Dec. 20, 2007

(54) Synthetic Focusing Method

(75) Inventor: Ray Andrew Simpkin, Auckland (NZ)

Correspondence Address: DANN, DORFMAN, HERRELL & SKILLMAN, 1601 MARKET STREET, SUITE 2400, PHILADELPHIA, PA, 19103-2307, US

(73) Assignee: INDUSTRIAL RESEARCH LIMITED, Birkenhead, Auckland (NZ)

(21) Appl. No.: 10/581,400

(22) PCT Filed: Sep. 12, 2005
(86) PCT No.: PCT/NZ05/00240

§ 371 Date: May 8, 2007

Related U.S. Application Data
(60) Provisional application No. 60/608,713, filed on Sep. 10, 2004.
(60) Provisional application No. 60/608,751, filed on Sep. 10, 2004.

(51) Int. Cl. A61B 5/05

(52) U.S. Cl. 600/407

(57) ABSTRACT

A method of generating a three-dimensional radar image of a body part having multiple image points. The method comprises receiving radiation information (11) obtained at an array of scan locations relative to the body part, surface profile information (12) relating to the body part, and estimates of body part properties (13). The method further comprises constructing each image point by: determining the minimum optical paths between each scan location and the image point based on the scan locations, surface profile information and body part properties; phase-shifting the radiation information based on the minimum optical paths to equalise the radiation information; and then summing the equalised radiation information to provide a value for the image point. The 3D radar image of the body part is then generated based on the values of each of the image points.

### FIELD OF THE INVENTION

The present invention relates to a synthetic focusing method for use in microwave medical imaging of body parts. In particular, although not exclusively, the synthetic focusing method may be utilised in microwave medical imaging applications such as breast cancer screening.

### BACKGROUND TO THE INVENTION

Breast cancer is the most common cancer to affect women. The detection of malignancies at an early stage is deemed to offer the best prognosis for patients and this has lead to the establishment of screening programmes aimed at early detection.

X-ray mammography is one commonly used breast cancer screening method due to its simplicity, high-resolution images and cost effective implementation. However, x-ray mammography has a number of associated limitations and drawbacks. X-rays are an example of ionizing electromagnetic radiation which can damage tissue and in some cases initiate malignant tumours. X-ray mammography requires the patient's breasts to be compressed between two plates which is uncomfortable for many women and makes it difficult to determine the true three-dimensional (3D) location of any suspicious features. Furthermore, women with silicone breast implants are also at risk from implant rupture due to the compression process. X-ray images are two-dimensional (2D) and a number of images from different views must typically be taken to provide some indication of the 3D location of suspicious features. X-ray detection of suspicious features relies on differences in density within the breast tissue under test and the density contrast between healthy and malignant breast tissue is small, typically only about 2%, which can make detection of tumours difficult. For post-menopausal women, x-ray mammography fails to detect up to 15% of cancers. For younger women, whose breast density is usually higher, up to 40% of cancers can be missed by x-ray mammography. Generally, the smallest tumour detectable with x-ray mammography is about 4 mm in diameter. A tumour this size is reckoned to have been in the body for about 6 years, that is, not particularly early in the tumour's development.

All of the above have provided significant incentive for researchers to develop alternative methods for breast cancer detection that alleviate some of the difficulties associated with x-ray mammography. Microwave imaging, which utilises electromagnetic waves in the microwave region, has been identified as having potential for improved detection of breast cancer due to the large difference in complex permittivity between healthy and malignant breast tissue. U.S. Pat. Nos. 4,641,659, 5,704,355, 5,807,257, 5,829,437, 5,920,285, 5,969,661, 6,061,589, 6,421,550, 6,448,788, 6,504,288, and international PCT patent application publication number WO 2004/073618 disclose various microwave imaging systems and associated techniques for focusing microwave energy.

It is an object of the present invention to provide an improved focusing method for microwave medical imaging of body parts, or to at least provide the public with a useful choice.

### SUMMARY OF THE INVENTION

In a first aspect, the present invention broadly consists in a method of generating a three-dimensional radar image of a body part having multiple image points comprising: receiving radiation information obtained at an array of scan locations relative to the body part, the radiation information being obtained at multiple microwave frequencies at each of the scan locations; receiving surface profile information relating to the body part; receiving estimates of body part properties; constructing each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and generating the 3D radar image of the body part based on the values of each of the image points.

Preferably, the body part properties may comprise: estimates of the thickness and dielectric constant of dielectric interfaces of the body part between the scan locations and the image point; and estimates of the dielectric constant of the body part in the vicinity of the image point.

Preferably, the body part properties may comprise: estimates of the thickness and dielectric constant of the skin dielectric interface; and the dielectric constant of the body part in the vicinity of the image point. More preferably, the body part may be a human breast and the body part properties may comprise: estimates of the thickness and dielectric constant of the skin dielectric interface of the breast; and the dielectric constant of the breast tissue.

Preferably, determining the minimum optical paths between each scan location and the image point being constructed may comprise: mapping the valid optical paths between each scan location and the image point using Snell's Law of Refraction and selecting the minimum optical path from the valid optical paths.

Preferably, the values of the image points may be radar intensity values.

Preferably, the method may further comprise displaying the three-dimensional radar image of the body part.

Preferably, the radiation information may be obtained at each scan location at multiple discrete frequencies of at least 10 GHz. More preferably, the radiation information may be obtained at multiple discrete frequencies in the frequency range of approximately 10 GHz-18 GHz.

Preferably, the radiation information may be obtained at at least 10 discrete frequencies.

Preferably, the radiation information may be obtained at at least 100 scan locations relative to the body part.

In a second aspect, the present invention broadly consists in a system for generating a three-dimensional radar image of a body part having multiple image points comprising: an input for receiving input data comprising: radiation information obtained at an array of scan locations relative to the body part, the radiation information being obtained at multiple microwave frequencies at each of the scan locations; surface profile information relating to the body part; and estimates of body part properties; a processor arranged to process the input data to construct each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and an output for sending output data relating to the image point values for the generation of the 3D radar image of the body part.

Preferably, the body part properties may comprise: estimates of the thickness and dielectric constant of dielectric interfaces of the body part between the scan locations and the image point; and estimates of the dielectric constant of the body part in the vicinity of the image point.

Preferably, the body part properties may comprise: estimates of the thickness and dielectric constant of the skin dielectric interface; and the dielectric constant of the body part in the vicinity of the image point. More preferably, the body part may be a human breast and the body part properties may comprise: estimates of the thickness and dielectric constant of the skin dielectric interface of the breast; and the dielectric constant of the breast tissue.

Preferably, the processor may be arranged to determine the minimum optical paths between each scan location and the image point being constructed by mapping the valid optical paths between each scan location and the image point using Snell's Law of Refraction and selecting the minimum optical path from the valid optical paths.

Preferably, the values of the image points may be radar intensity values.

Preferably, the system may further comprise an output display for receiving the output data and displaying the three-dimensional radar image of the body part.

Preferably, the radiation information may be obtained at each scan location at multiple discrete frequencies of at least 10 GHz.

Preferably, the radiation information may be obtained at multiple discrete frequencies in the frequency range of approximately 10 GHz-18 GHz.

Preferably, the radiation information may be obtained at at least 10 discrete frequencies.

Preferably, the radiation information may be obtained at at least 100 scan locations relative to the body part.

In a third aspect, the present invention broadly consists in a computer program for generating a three-dimensional radar image of a body part having multiple image points, the program being arranged to: receive input data comprising: radiation information obtained at an array of scan locations relative to the body part, the radiation information being obtained at multiple microwave frequencies at each of the scan locations; surface profile information relating to the body part; and estimates of body part properties; process the input data to construct each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and output data relating to the image point values for the generation of the 3D radar image of the body part.

Preferably, the body part properties may comprise: estimates of the thickness and dielectric constant of dielectric interfaces of the body part between the scan locations and the image point; and estimates of the dielectric constant of the body part in the vicinity of the image point.

Preferably, the body part properties may comprise: estimates of the thickness and dielectric constant of the skin dielectric interface; and the dielectric constant of the body part in the vicinity of the image point. More preferably, the body part may be a human breast and the body part properties may comprise: estimates of the thickness and dielectric constant of the skin dielectric interface of the breast; and the dielectric constant of the breast tissue.

Preferably, the computer program may be arranged to determine the minimum optical paths between each scan location and the image point being constructed by mapping the valid optical paths between each scan location and the image point using Snell's Law of Refraction and selecting the minimum optical path from the valid optical paths.

Preferably, the values of the image points may be radar intensity values.

Preferably, the computer program may output data to an output display for displaying the three-dimensional radar image of the body part.

Preferably, the radiation information may be obtained at each scan location at multiple discrete frequencies of at least 10 GHz.

Preferably, the radiation information may be obtained at multiple discrete frequencies in the frequency range of approximately 10 GHz-18 GHz.

Preferably, the radiation information may be obtained at at least 10 discrete frequencies.

Preferably, the radiation information may be obtained at at least 100 scan locations relative to the body part.

The term ‘comprising’ as used in this specification and claims means ‘consisting at least in part of’, that is to say when interpreting statements in this specification and claims which include that term, the features, prefaced by that term in each statement, all need to be present but other features can also be present.

The invention consists in the foregoing and also envisages constructions of which the following gives examples only.

### BRIEF DESCRIPTION OF THE DRAWINGS

Preferred forms of the invention will be described by way of example only and with reference to the drawings, in which:

FIG. 1 is a flow diagram showing the generic process of generating a three-dimensional radar image using the synthetic focusing method of the present invention;

FIG. 2 is a flow diagram showing the generic synthetic focusing method of the present invention for constructing each image point of the three-dimensional radar image;

FIG. 3 is a flow diagram showing the process of calculating minimum optical paths between scan locations and image points;

FIG. 4 is a schematic diagram showing an example implementation of the synthetic focusing method of the invention for generating a three-dimensional radar image of a human breast;

FIG. 5 is a perspective view of a microwave medical imaging system that employs the synthetic focusing method of the invention to generate three-dimensional radar images of breasts;

FIG. 6 is a perspective view of a sensor head of the microwave medical imaging system of FIG. 5;

FIG. 7 is a block diagram of the microwave medical imaging system of FIG. 5;

FIG. 8 is a block diagram of the radar device of the microwave medical imaging system of FIG. 5;

FIG. 9 shows a two-dimensional image slice through a three-dimensional radar image of a breast that was generated by a prototype microwave medical imaging system using the synthetic focusing method of the invention in a pre-clinical trial on a patient;

FIGS. 10a and 10b show x-ray mammograms, from craniocaudal and mediolateral oblique views respectively, of the same breast of the patient in the pre-clinical trial referred to in relation to FIG. 9; and

FIG. 11 shows the prototype breast imaging system used in the pre-clinical trial referred to in relation to FIG. 9.

### DETAILED DESCRIPTION OF PREFERRED EMBODIMENTS

The synthetic focusing method may be implemented in a microwave medical imaging system to generate three-dimensional radar images of body parts. The synthetic focusing method may be implemented in software on a computer or processor, or as a program on a programmable device, or may be implemented using any other electronic means.

In the preferred form, the synthetic focusing method is integrated into a microwave medical imaging system and is designed to generate 3D radar images of body parts for diagnostic purposes. The synthetic focusing method is a post-processing technique of focusing radiation information obtained from a radar device to progressively generate a 3D radar image.

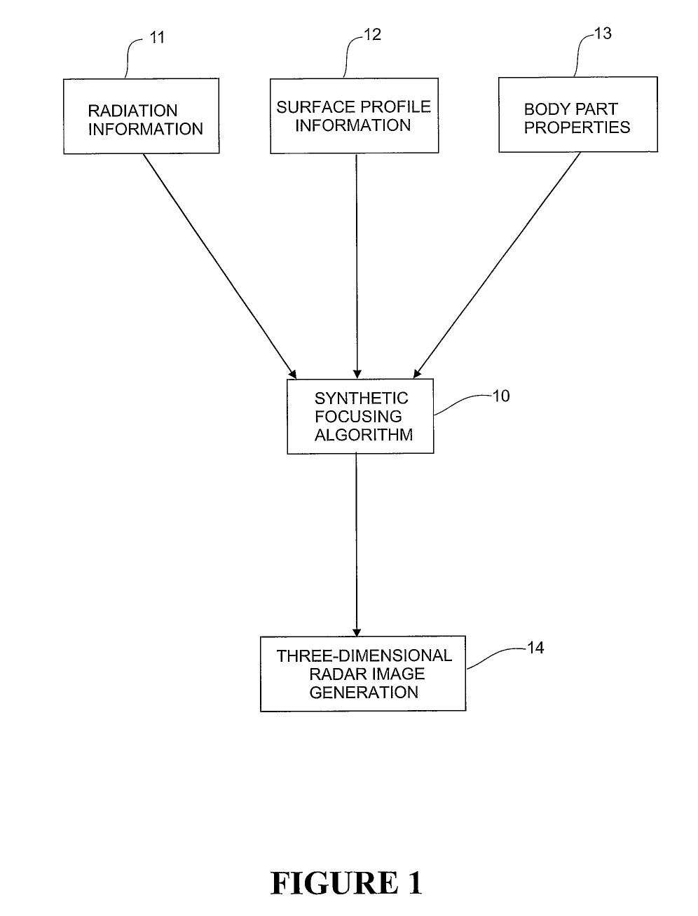

Referring to FIG. 1, the preferred form synthetic focusing method is implemented in the form of a software data processing algorithm. The synthetic focusing algorithm 10 processes three sets of input data, namely radiation information 11, surface profile information 12, and body part properties 13, to generate output in the form of a 3D radar image 14 for display. In particular, the synthetic focusing algorithm 10 receives radiation information 11 obtained at a large number of scan locations relative to the body part at multiple discrete frequencies and then focuses that information, using surface profile information 12 relating to the body part along with knowledge or estimates of body part properties 13 such as dielectric constants of the body part and skin thickness, at multiple image points within the body part to progressively build up a 3D radar image 14.

The radiation information 11 may be obtained by a radar device that scans the body part with microwave energy. More specifically, the radar device is arranged to transmit broadband microwave radiation into the body part and then receive radiation reflected back from the body part at an array of scan locations relative to the body part. The radiation information obtained at each of the scan locations is the amplitude and phase of the reflection coefficient of the reflected radiation received. The radar device may utilise synthetic of real aperture techniques to obtain the radiation information. Further, the radar device is arranged to obtain radiation information at multiple discrete frequencies at each of the scan locations.

The surface profile information 12 is the 3D geometric external skin surface profile of the body part being imaged. This information may be obtained by a three-dimensional profiler using laser time-of-flight, laser triangulation, ultrasound, broadband microwave signals, image-based techniques or any other 3D profiling methodology.

The body part properties 13 relate to the various dielectric interfaces of the body part being imaged. For example, estimates or knowledge of the skin dielectric interface is required. In particular, knowledge or estimates of skin thickness and skin dielectric constant at the microwave frequencies are required. The thickness and dielectric constant of any other dielectric interface of the body part between the skin and the image points to which the radiation information is being focused must also be known or estimated. Knowledge or estimates of the dielectric constant in the vicinity of the image points is also required. The body part properties assist in the mapping of radiation information through the body part.

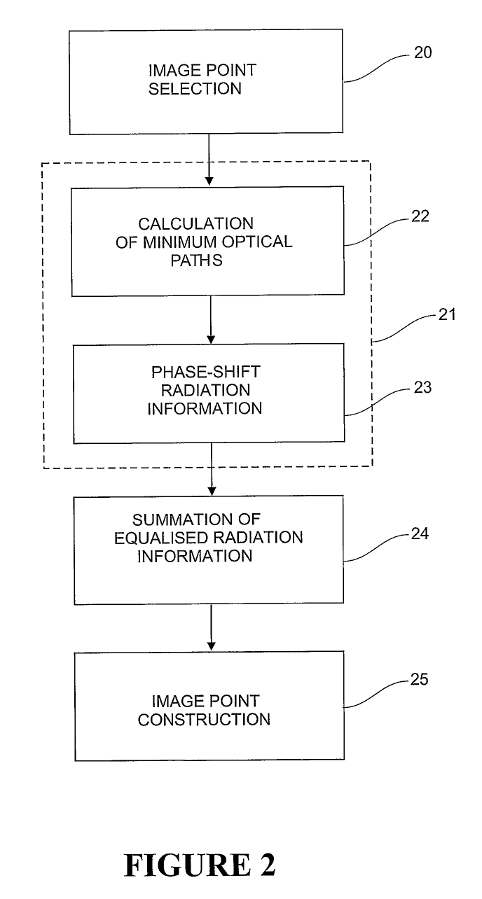

As mentioned, the synthetic focusing algorithm 11 generates the 3D radar image of the body part on a point-by-point basis by progressively synthetically focusing the radiation information to each image point. Referring to FIG. 2, the process of constructing each image point in the 3D radar image will be explained. Firstly, an arbitrary image point is selected within the 3D profile of the body part to be imaged and the 3D location of the arbitrary image point is stored. A two-staged equalisation process 21 is then undertaken. The first step involves the calculation or determination of the minimum optical paths between each scan location and the image point. The second step 23 involves phase-shifting the radiation information from each of the scan locations based on the values of their respective minimum optical paths to the image point. Once the phase-shift has been calculated and applied to the radiation information at scan locations, the equalised radiation information is then summed 24 over all scan locations and all discrete frequencies. At the completion of the summation step 24, the radiation information from each of the scan locations is synthetically focused to a focal point that coincides with image point. The image point is then constructed 25 based on the synthetically focused data. In particular, the magnitude of the focused data can be converted into an radar intensity value for the image point.

The steps 20-25 are carried out for each image point to progressively build up a 3D radar image. The resolution of the 3D radar image is typically dictated by the microwave frequencies at which the radiation information (radar data) was obtained.

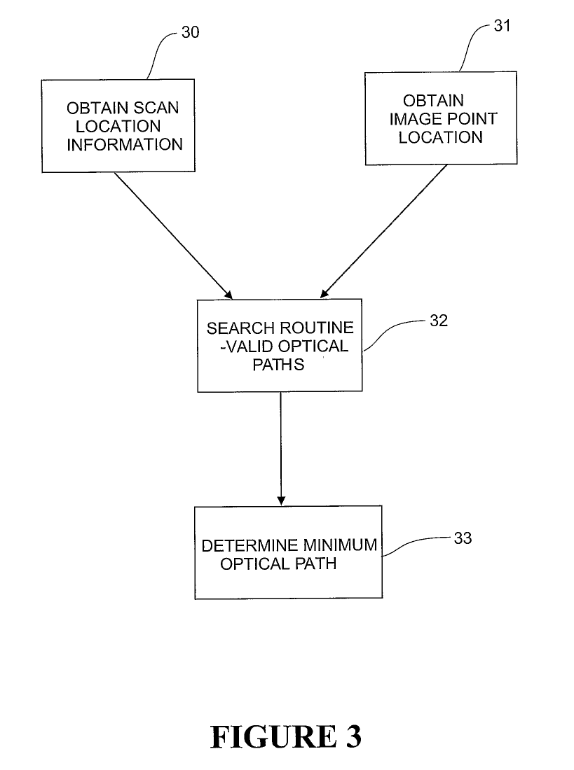

The process of calculating minimum optical paths (step 22) between the scan locations and the image points will now be described with reference to FIG. 3. In particular, the algorithm for determining the minimum optical path between a particular scan location and an image point will be described. Firstly, information about the 3D scan location 30 and the 3D location of the image point 31 are obtained. A search routine 32 is then implemented to determine the possible valid optical paths of radiation between the scan location and the image point. The search routine 32 determines valid optical paths by progressively mapping the optical paths of radiation from the scan location and into the body part through different positions on the external skin surface of the body part. Valid optical paths are those that travel from the scan location through the body part to the image point. The mapping of the optical paths is conducted using Snell's Law of Refraction. In particular, to map the optical path of radiation through the body part for a given scan location and skin surface point requires knowledge or estimates of the dielectric constant of the skin, skin thickness and the dielectric constant in the vicinity of the image point, along with knowledge or estimates of the thickness and dielectric constant of any other dielectric interfaces along the path of radiation between the scan location and image point. More specifically, the mapping process utilises body part properties along with information on the scan location and skin surface location to map the optical path of radiation through the body part using Snell's Law of Refraction. Once the search routine is complete, the minimum optical path 33 is computed from the knowledge of the possible valid optical paths.

The resolution of the 3D radar image produced by the synthetic focusing method is dependent on the nature of the input data. For example, the frequency range and number of discrete frequencies and scan locations at which the radiation information is obtained will dictate the resolution of the 3D radar image produced. The accuracy of the surface profile information and the body part properties may also affect the resolution and quality of the 3D radar image.

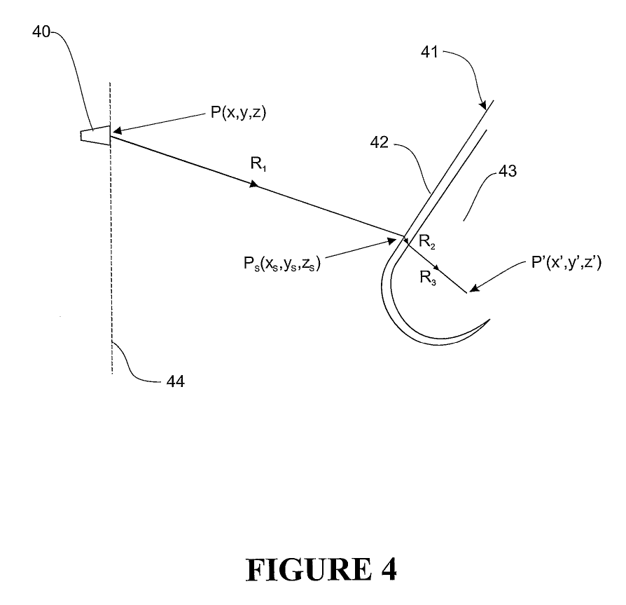

Referring to FIG. 4, an example of the synthetic focusing method applied to microwave breast imaging will be described, but it will be appreciated that the synthetic focusing method can be adapted for other body parts. In this example, the synthetic focusing method is utilised to generate 3D radar images of a human breast that has been scanned by an imaging system to obtain radiation information and surface profile information about the breast. In particular, the imaging system gathers reflection coefficient data (radiation information) from the breast over a range of microwave frequencies at multiple scan locations (antenna locations), along with surface profile information. The synthetic focusing method is utilised by the imaging system to generate images of scattered field intensity (3D radar images) of the scanned breast by post-processing the data obtained by the imaging system. An example of the imaging system will be described in more detail later.

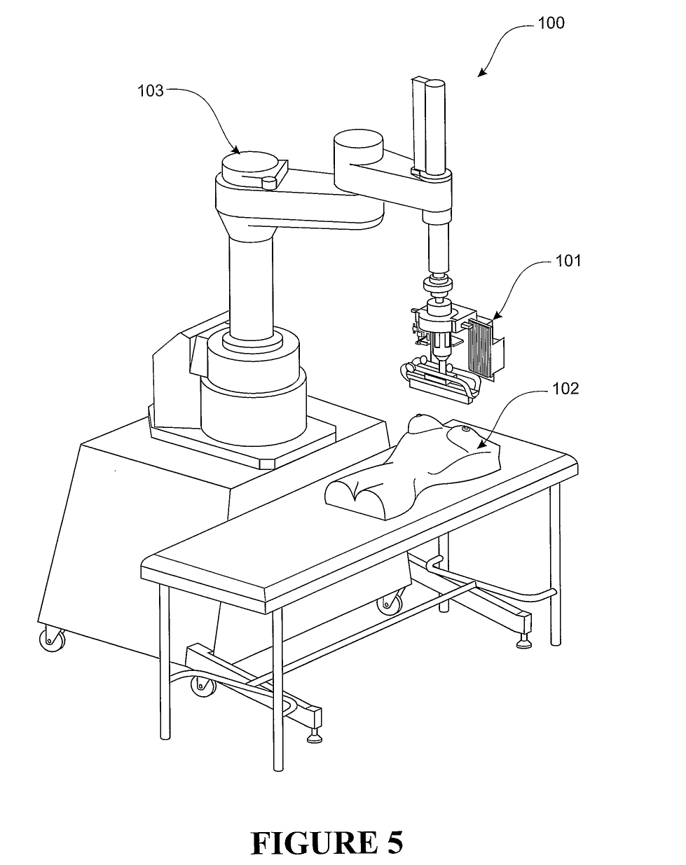

FIG. 4 shows the geometry of an antenna and breast configuration in a 3D Cartesian coordinate system. By way of example, one antenna 40 is shown at one of the scan locations in a synthetic aperture, S. The breast 41 is defined by skin 42 and breast interior tissue 43. The vector R₁ extends from the antenna point denoted P(x,y,z) in the antenna measurement plane 44 (defined by synthetic aperture, S) to a surface point on the outer surface of the breast denoted by Pₛ(xₛ,yₛ,zₛ). The vector R₂ extends from this surface point on the outer skin surface to a point on the interior skin surface. The vector R₃ extends from this interior skin surface point to the image point P′(x′,y′,z′), the point at which microwave energy is to be focused. This image point can be chosen arbitrarily. However, the path mapped out by the vectors R₁, R₂ and R₃ between antenna point and image point is not defined in an arbitrary fashion. Fermat's Principle is invoked so that the optical path is the minimum one possible. The minimum optical path, Rₘᵢₙ, is defined as follows for the geometry of FIG. 5:
Rₘᵢₙ=Minimum Value of {|R₁|+\sqrt{εₛₖᵢₙ}|R₂|+\sqrt{εₜᵢₛₛᵤₑ}|R₃|} (1)
where

εₛₖᵢₙ=Dielectric constant of skin.

εₜᵢₛₛᵤₑ=Dielectric constant of breast tissue.

There is one minimum path Rₘᵢₙ for each image point and antenna point (scan location). So, for a given point in the image, there is a set of N Rₘᵢₙ values where N is the number of antenna points (scan locations) used in the synthetic aperture.

The scattered electric field vector measured by the antenna at the point P(x,y,z) at a frequency denoted by the free-space propagation constant, k, is defined as E_{scat}(x,y,z,k).

The free-space propagation constant, k, is given by 2π/λ where λ is the free-space wavelength. A planar synthetic aperture is used here so that z=constant on the measurement plane.

The 3D radar image at a given point P′ is now formed by applying a phase shift equal to 2kRₘᵢₙ to the measured reflection coefficient data for each point (scan location) in the synthetic aperture and then summing over all antenna locations. Summing over the frequency domain is also carried out. If the dielectric properties of the skin and breast tissue are assumed to vary negligibly with frequency (which is a good approximation), then the minimum paths between each image point and all antenna points will not depend on frequency. Therefore, once the minimum paths have been computed for a given combination of image point and antenna points, they can be used for all frequencies in the summation over the frequency domain.

Mathematically, the above process can be represented by the following three-fold integral for generating the image, I, at P′(x′,y′,z′):

$$I ( {x}^{′} , {y}^{′} , {z}^{′} ) = {\int }_{S} {\int }^{{k}_{2}} {\int }_{{k}_{1}} {E}_{scat} ( x , y , z , k ) {ⅇ}^{2 j {kR}_{min}} ⅆ S ⅆ k \qquad ( 2 )$$

where

S=Synthetic aperture area.

k₁=Free-space propagation constant at lowest frequency.

k₂=Free-space propagation constant at highest frequency.

In equation (2) the factor of 2 in the phase shift term is present due to the need to account for the two-way path ‘there and back’ between antenna and image point. This phase shift term equalises the phase of the received signals from a given image point at all antenna locations so that when the summation over the synthetic aperture takes place, all quantities add up in phase to produce a much enhanced field at the image point location. The measured fields are therefore focused at the image point. This is an example of synthetic focusing applied to an antenna array.

The use of the minimum optical path Rₘᵢₙ to calculate the appropriate phase shift is consistent with the Method of Stationary Phase often used to evaluate integrals of the type given in equation (2). This type of integral is characterised by a phase function in the integrand—often expressed as a complex exponential like that in (2)—which is a function of the integration variables. For values of the phase function which are varying rapidly with position, the oscillatory nature of the integrand in these regions results in a negligible contribution to the integral since positive and negative going portions of the oscillatory phase function tend to cancel each other out. The only significant contribution to the value of the integral comes from the region where the phase function is varying slowly such as in the vicinity of a stationary point in the phase function. This region corresponds to the minimum path Rₘᵢₙ and this is why it is used in the phase function exp(2jkRₘᵢₙ) of the integrand in (2).

The vector nature of the electric field in (2) has been ignored since the dominant scattered field component will be co-polarised with the dominant polarisation present in the aperture of the antenna. That is, de-polarisation effects are ignored in the focusing algorithm—these will not be significant for a monostatic reflection coefficient measurement system.

Equation (2) appears simple in form but complexity lies in the need to determine the values of Rₘᵢₙ for each combination of image point and antenna point (scan location). The determination of Rₘᵢₙ can be performed as a separate computational exercise and need only be computed once for a given antenna and breast geometry. In order to determine Rₘᵢₙ, it is necessary to have knowledge of the following:
the geometric profile of the breast's outer surface relative to some known origin.
an estimate of the dielectric constants of the skin and interior breast tissue.
an estimate of the skin thickness.

In the imaging system, the geometric profile of the breast's outer surface is measured by, for example, a 3D laser profiler. Knowledge of skin thickness and dielectric constant of the skin and breast tissue to a high degree of accuracy is not necessary. An accepted value for the dielectric constant of skin at frequencies in the range 10 GHz to 18 GHz is 40 and that of the interior breast tissue is 9. The skin thickness may be nominally taken as 2 mm. Values within 10% of the true values for dielectric constant will give rise to 5% errors in the optical path calculation due to the square-root dependence on the dielectric constants (see equation (1)). The skin can be considered as being a dielectric interface between the air and breast tissue through which the radiation travels.

For imaging purposes, the breast interior is assumed to be a homogeneous medium with a (mean) dielectric constant of εₜᵢₛₛᵤₑ. While the breast interior will not be homogenous in practice, deviations from this mean dielectric constant will not be large for normal breast tissue. Large deviations from this ‘background’ dielectric constant—such as encountered with malignant tumours—will show up readily in the radar image whereas the smaller deviations in dielectric properties normally encountered with healthy breast tissue will scatter weakly and not show up as significant features in the radar image. Typically the imaging system will operate as a breast screening tool aimed at detecting the presence of suspicious objects within the breast rather than as a diagnostic tool. The above assumption of homogeneity for the breast interior is deemed sufficient for screening purposes.

The minimum path Rₘᵢₙ is a function of the breast geometry as well as the antenna geometry and will therefore be unique to a particular patient. Values of Rₘᵢₙ are calculated by fixing the antenna and image point locations and varying the position of the point Pₛ on the skin's outer surface until the minimum value of the optical path is found. The two variables of interest here are xₛ and yₛ, the x and y coordinates on the outer surface of the skin. The value of zₛ is governed by the outer surface profile data (as measured by the laser system) and is a function of xₛ and yₛ.

For a given point on the skin's outer surface, the point on the inner surface of the skin (where it meets the interior breast tissue) is automatically defined by Snell's Law of Refraction and so the vectors R₁, R₂ and R₃ are all fully defined for given values of antenna and image points along with values of xₛ and yₛ. Snell's Law of Refraction is wholly consistent with Fermat's Principle for a minimum optical path. Thus, the only variables in the search routine for the minimum path are xₛ and yₛ.

Once found, the values of minimum path Rₘᵢₙ are stored in a five-dimensional array. Two indices are used to define the antenna location in the synthetic aperture and a further three to define the image point in 3D space. Image generation then proceeds by the numerical evaluation of the integral in equation (2). The image itself is usually displayed as the magnitude of the image function I(x′,y′,z′).

Use of commercially available 3D visualisation software is the most effective means of displaying the 3D radar image data. Iso-surfaces and volume rendering visualisations are particularly appropriate for detecting suspicious features within the breast.

An example of an imaging system that would employ the synthetic focusing method will now be described with reference to FIGS. 5-8. The imaging system is a microwave medical imaging system for body parts and will be described in the context of breast scanning by way of example. The imaging system is arranged to scan a patient's breasts with microwave radiation in order to generate 3D radar images of each breast which can be examined for suspicious features such as malignant tumours. There is a large difference in complex permittivity between healthy and malignant breast tissue and this leads to greater scattered field amplitudes from malignant tumours embedded in healthy tissue which show up readily in a microwave image of scattered field intensity. For example, the real part of complex permittivity (the dielectric constant) for a malignant tumour is of the order of 50 at a frequency of 10 GHz whereas healthy tissue has a value of about 9. Hence, radar images are suited to breast tumour detection since the high permittivity contrast between malignant and healthy tissue translates to high-contrast images.

The imaging system generates 3D radar images based on the intensity of the scattered field as a function of position from measurements of scattered fields external to the breast. The imaging system utilises the synthetic focusing algorithm of the invention to provide coherent addition of scattered fields at a given image point within the 3D radar image, thereby giving a measure of the scattered field intensity at a point in the breast being scanned.

Referring to FIG. 5, the preferred form imaging system 100 includes a sensor head 101 that is translated relative to a patient 102 by a robot 103. The imaging system is arranged to scan each of the patient's exposed breasts individually and generate respective 3D radar images. In particular, the imaging system scans the patient's breasts to simultaneously obtain radiation information and surface profile information which are processed by an image generation algorithm to generate the 3D radar images. The preferred form sensor head 101 does not make contact with the patient 102 and there is no coupling medium, other than air, between the patient and sensor head during scanning. In an alternative form of the imaging system, the sensor head 101 could be moved by means other than robot 103. It will also be appreciated that the patient could be moved relative to a stationary sensor head in another alternative form of the imaging system. For example, the imaging system may have a moveable support, platform or bed that supports the patient and is operable to move them past the sensor head of the imaging system during the scan.

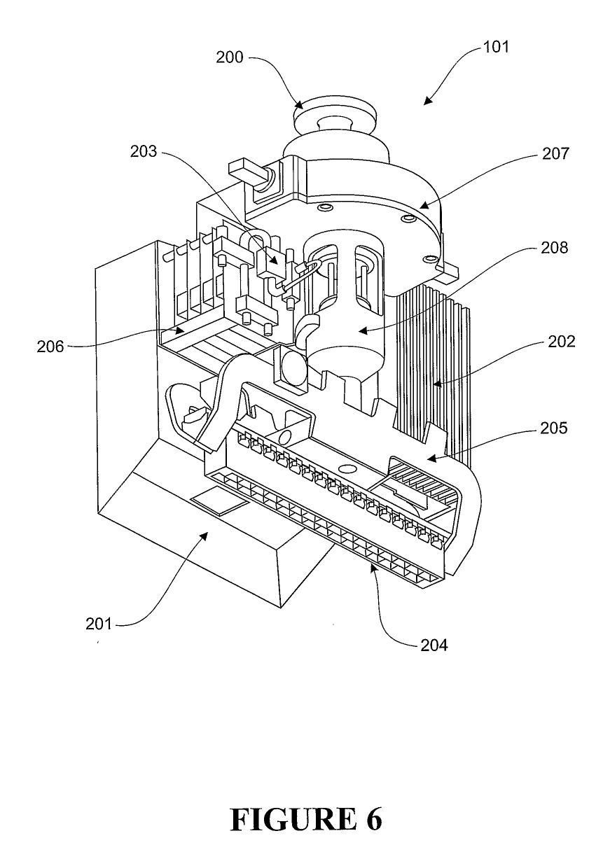

Referring to FIG. 6, the sensor head 101 is mounted to the robot scanning mechanism in the preferred form by a mounting flange 200. The sensor head includes a 3D profiler 201 that is arranged to obtain geometric surface profile information of the breast during scanning. In the preferred form, the 3D profiler is a laser profiler device which uses a scanning laser stripe and charge-coupled device (CCD) sensor to provide range information by triangulation. The laser output power from the 3D profiler is deemed eye-safe. It will be appreciated that other types of 3D profiling devices could be utilised to obtain geometric surface profile information about the breasts. For example, alternative forms of 3D profilers may utilise ultrasound or broadband microwave signals to obtain the surface profile information. Other examples of 3D profilers that may be employed in the imaging system are laser based time-of-flight systems or image-based systems. Other means of obtaining geometric information about an arbitrary shape, such as a human breast, are known to those skilled in the art and could also be utilised in the imaging system if desired.

The sensor head 101 also carries a radar device that is arranged to transmit non-ionizing radiation toward the breast and then receive radiation reflected back from the breast at multiple predetermined scan locations relative to the breast. The radar device includes a radiation source 202 and receiver 203 that are connected to an array 204 of antenna elements or waveguides via a switching network 205. In the preferred form, the radiation source 202 is a Yttrium Iron Garnate (YIG) oscillator that generates microwaves over a broad range of frequencies and the radiation receiver 203 is a six-port reflectometer. The radar device is operated and controlled by an on-board computer system 206 and also has a calibration device 207 and an associated servo-motor 208.

The preferred form radar device is arranged to obtain radiation information at an array of scan locations that define a synthetic aperture relative to the patient's breast. The radar device sweeps out the synthetic aperture by translating the antenna array 204 within the synthetic aperture and sequentially operating each of the individual antenna elements to obtain radiation information at the multiplicity of scan locations. For example, the preferred form radar device has a linear array of thirty two antenna elements arranged in two rows of sixteen antenna elements. During scanning, the antenna array is, for example, translated mechanically by the robot scanning mechanism to thirty two equally spaced locations in an orthogonal direction relative to the antenna array. At each of the thirty two locations, the thirty two individual antenna elements are sequentially connected to the radiation source and receiver by the switching network so that radiation information can be obtained at each of the 1024 scan locations of the synthetic aperture. The number of scan locations may vary depending on the design requirements. Preferably there are at least 100 scan locations, more preferably at least 500 scan locations, and even more preferably at least 1024 scan locations. Ultimately, the number of scan locations must be sufficient to enable the generation of a reasonable 3D radar image and will depend on other design parameters such as aperture size, antenna element spacing, frequency range, amount of radiation data required etc.

The preferred form array of scan locations is linear in nature with the scan locations being arranged in rows and columns along a plane with regular interspacing. However, it will be appreciated that the array of scan locations does not necessarily have to be linear or regular with respect to interspacing between scan locations. The array of scan locations may be irregular in shape and there may be variable interspacing between scan locations. Furthermore, the scan locations do not necessarily have to lie along the same plane.

In the preferred form, the antenna array has monostatic antenna elements, i.e. the antenna elements both transmit and receive microwave signals, but separate transmit and receive elements could be used in an alternative bistatic arrangement.

The size of the synthetic aperture should preferably be no less than twice that of the body part to be imaged, so that the body part is illuminated sufficiently well by electromagnetic radiation from each antenna element. For imaging a breast, a value of 15 cm has been assumed as a typical linear dimension. Therefore, the minimum synthetic aperture size, D, is preferably twice this value, namely 30 cm along each transverse axis. It will be appreciated that imaging system can alternatively operate with a smaller synthetic aperture to body part ratio depending on the system requirements.

The required antenna element spacing in the antenna array is determined from the requirement to satisfy the Nyquist sampling criterion at the highest frequency of operation (shortest wavelength) so that grating lobes are avoided in the resulting image. This criterion requires that the element spacing be no greater than one half of a wavelength at the highest frequency of operation. For example, an upper frequency limit of 18 GHz gives the largest allowed element spacing as 8.3 mm. This element spacing in turn dictates the number of predetermined antenna scan locations in the synthetic aperture when combined with the minimum synthetic aperture size.

The radiation information at each scan location within the synthetic aperture is obtained by illuminating the breast with microwave radiation from a transmitting antenna and then measuring the amplitude and phase of the reflected wave (scattered field) from the breast. In the preferred form imaging system, the radiation information is obtained at each scan location by repeating the measurement over a broad range of frequencies, one frequency at a time. For example, the imaging system utilises broadband microwave energy at a multiplicity of discrete frequencies over a predetermined range of the microwave band. In the preferred form radar device, a six-port reflectometer is incorporated into the microwave signal path. The six-port reflectometer is arranged to produce four voltages from diode detectors connected to its output ports from which it is possible to determine the amplitude and phase of the reflected signals relative to the incident (transmitted) signal.

It will be appreciated that there are other alternative antenna arrangements which could be utilised to obtain the radiation information at each of the scan locations within the synthetic aperture. For example, the radar device may be equipped with only a single antenna element that is translated mechanically to all scan locations with the synthetic aperture, although such an arrangement would be slow in terms of data acquisition speed. As mentioned, an alternative form of the imaging system may involve the patient being automatically moved past a stationary sensor head during the scan. The sensor head may utilise an array of antenna elements or a single antenna element to obtain radiation information at each of the multiplicity of predetermined scan locations of the synthetic aperture as the patient is moved past the sensor head in a predetermined path by an operable moveable support. The essential requirement of the synthetic aperture arrangements mentioned is that there is relative movement between the antenna element(s) of the sensor head and the patient such that radiation information can be obtained at a multiplicity of locations relative to the patient's breast to sweep out the synthetic aperture. In another possible synthetic aperture approach, both the patient and antenna element(s) could be arranged to move relative to each other during the scan.

In an alternative form of the imaging system, a real aperture could be provided in which there is an antenna element at each of the predetermined scan locations over the breast. With a fixed, real aperture the radiation information is obtained by sequentially operating each antenna element one at a time. This arrangement does not require any relative movement between the sensor head and the patient. While a real aperture arrangement would be fast from a data acquisition viewpoint, it would also be more costly. The preferred form radar device utilises a synthetic aperture arrangement that is a compromise between data acquisition time and cost.

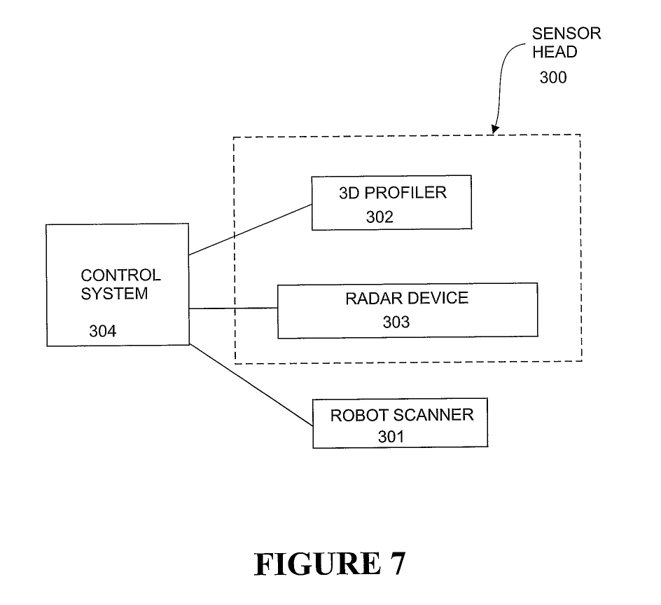

Referring to FIG. 7, the sensor head 300 is mounted to a robot scanning mechanism 301 that carries both the 3D profiler 302 and radar device 303. The robot scanning mechanism 301 is arranged to move the sensor head 300 relative to a patient's breast while the 3D profiler 302 and radar device 303 obtain surface profile information and radiation information respectively as described above. A control system 304 is provided that controls the robot scanning mechanism 301, 3D profiler 302 and radar device 303 during the breast scan. Further, the control system 304 is arranged to process the surface profile and radiation information to generate the 3D radar image of the breast. By way of example, the control system 304 may comprise a computer, such as a PC or laptop, upon which a graphical user interface (GUI) runs. The GUI may be operated by a user to control the imaging system. The control system 304 may also run the synthetic focusing algorithm to generate and display the 3D radar image, although it will be appreciated that a separate processing device may be utilised.

Preferably, the surface profile information and radiation information are obtained simultaneously during one scan of the patients breasts by the sensor head 101. However, simultaneous operation is not essential to the imaging system as sequential scans to obtain the radiation information and surface profile information could alternatively be implemented by the imaging system provided the patient remains relatively still between each scan. For example, the imaging system may be arranged to obtain surface profile information from a first scan in which only the 3D profiler 302 is operated and then radiation information may be obtained from a second scan in which only the radar device 303 is operated, or vice versa. It will be appreciated that a dual scanning system could utilise independently moveable sensors heads i.e. a 3D profiler sensor head and a radar device sensor head.

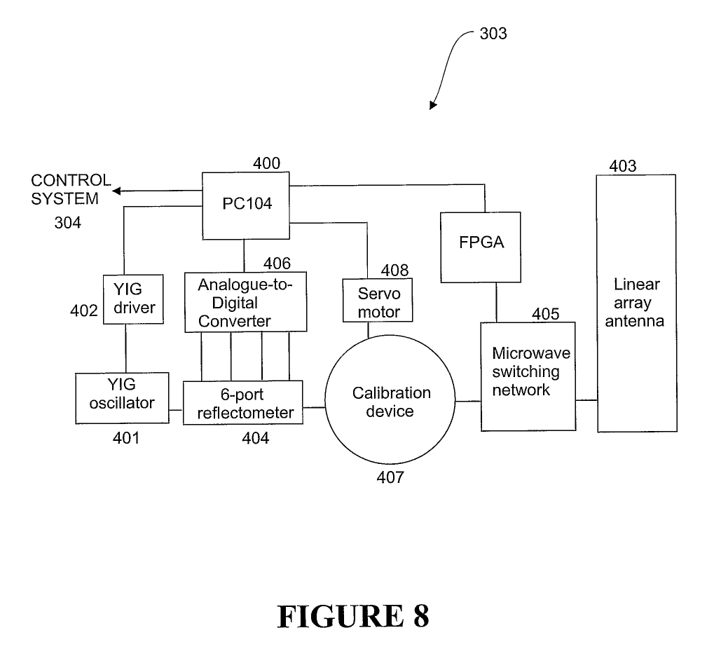

Referring to FIG. 8, the configuration and operation of the radar device 303 will be explained in more detail. The radar device 303 communicates with the control system 304 via an on-board computer system 400. The radar device has a YIG oscillator 401 which is operated in a swept frequency mode via its driver circuit 402 to generate microwave radiation at a large number of desired discrete frequencies. The driver circuit 402 is in turn controlled by a sequence of binary signals from the on-board computer system 400.

An important feature of the radar device is that the microwave power level emitted by each antenna element in the antenna array 403 is low and is of a non-ionizing nature. For example, the microwave power output from the YIG oscillator 401 may vary from 30 mW-50 mW depending on the frequency. However, the power level made available to each radiating element in the antenna array 403 may be in the order of 0.1 mW due to attenuation in the six-port reflectometer 404 and switching network 405. The sensor head 303 is also displaced, for example approximately 30cm, away from the patient's body which further reduces radiation exposure to the patient. Therefore, from a radiological stand point, the radar device is inherently safe.

The stand-off distance is not critical but should preferably be greater than five wavelengths at the lowest frequency of operation so that the illuminating wavefront from each antenna element has a spherical phase front with local plane-wave characteristics. That is, the breast is far removed from the reactive near-field region of the antenna and is illuminated by a wavefront having predictable phase and amplitude characteristics. A stand-off distance of ten wavelengths at the lowest frequency of operation is most preferable for reducing the effects of multiple reflections between breast and antenna, which can contaminate the measured data and subsequent radar images. The stand-off distance is a compromise between being large enough to satisfy the above criteria and small enough that the transmitted and received signal levels are not too low due to the space-attenuation factor (that is the 1/R⁴ dependence on the received power level, R being the object-antenna separation). This effect is compensated for in the preferred form by using a large number of elements in the synthetic aperture to enhance the received power levels when applying synthetic focusing. In addition, during the synthetic focusing process (which will be described later), the size of the focal spot is also degraded (i.e. becomes larger) as the object-antenna separation is increased. To this end, it is desirable to maintain a focal ratio of the order of unity in determining the appropriate stand-off distance.

A non-contact sensor head 300 enables the reflected signals from the breast to be accurately measured and allows calibration of the antenna system of the radar device in isolation. As mentioned, the stand-off distance between the breast and the plane of the synthetic aperture should preferably be at least 10 wavelengths at the lowest frequency of operation in order to reduce the effects of multiple reflections between antenna and breast to a negligible level. This allows the effects of the antenna system to be subtracted from the measured radiation information with the breast in place to give just the reflectivity of the breast in isolation. A typical stand-off distance used for the breast imaging device is therefore 30 cm at a minimum operating frequency of 10 GHz.

The radiation information to be measured by the radar device is the reflection coefficient of the reflected microwave signals at each location within the synthetic aperture and at each frequency of interest. In particular, the phase and amplitude of the reflection coefficient is measured. The six-port reflectometer 404 within the microwave signal path produces four voltages from diode detectors connected to its output ports from which the amplitude and phase of the reflected signals relative to the incident (transmitted) signal is determined.

The six-port reflectometer 404 essentially combines the reflected microwave signal from the breast under test with a portion of the incident wave. This is done using four different relative phase differences introduced by the six-port reflectometer 404 between incident and reflected waves. The four combinations of microwave signals are then sent to four square-law detector diodes that generate four output voltages. One of the four output voltages is used as a reference such that three voltage ratios are derived for each measurement. These three ratios are converted into the real and imaginary parts of the reflection coefficient. The measured reflection coefficient information is then converted into digital data by an analogue-to-digital converter 406 which in turn sends the digital data to the on-board computer system 400.

The radar device employs a near-field imaging method in that the distance between the antenna elements and the patient's breast has a focal ratio typically in the order of unity. Therefore, the transmitted wavefronts illuminating the breast are highly curved. Further, the imaging system utilises an image generation algorithm that images objects embedded in the breast interior. In particular, the image generation algorithm takes into account the refraction at the various dielectric interfaces in order to focus effectively within the breast.

In the preferred form the radar device 303 includes a calibration device 407 and associated servo-motor 408 that are arranged to calibrate the six-port reflectometer 404 and antenna system. Calibration of the six-port reflectometer 404 will be described first. In order to accurately determine the complex reflection coefficient from the voltage outputs of the six-port reflectometer 404, it is necessary to calibrate the reflectometer to account for any imperfections and idiosyncrasies in the componentry. A number of ‘calibration standards’ are connected to the measurement port of the reflectometer and output voltages acquired as per a normal measurement. The calibration standards have known reflection coefficients for all frequencies of interest. For example, for the preferred form radar device, nine standards are used, all of them different lengths of short-circuited rectangular waveguide.

It is possible to calibrate a six-port reflectometer using only five standards. However, a total of nine are made available in the preferred form imaging system due to the broad range of frequencies used. The key to an accurate calibration procedure for a six-port reflectometer is the selection of five standards with widely spaced reflection coefficient phase angles (the magnitude of the reflection coefficient is unity for all short-circuit standards). Having nine standards available allows one to select the five best phase angles for use at a given frequency thereby maintaining accurate calibration across the whole frequency band.

The waveguide standards are built into the rotary calibration device 407, mounted on the sensor head, that is able to connect each standard to the reflectometer measurement port one at a time by means of a servo-motor 408.

For each of the nine calibration standards, the four six-port reflectometer output voltages are measured for each frequency in the band and stored. These are converted into real and imaginary parts of reflection coefficient and a set of calibration coefficients generated using a standard algorithm (not described here). The calibration coefficients characterise the six-port reflectometer 404 and enable the reflection coefficient of a breast under test to be accurately determined from the four diode detector output voltages taking into account the imperfections in the reflectometer 404 itself.

The calibration of the antenna system will now be described. In order to extract the amplitude and phase of the reflection coefficients attributable purely to the patient's breast under test it is necessary to remove the contribution from the antenna system. This is done by performing a series of reflection coefficient measurements on the antenna system with no patient present. In particular, two measurements are carried out on the antenna system as outlined below.

First measurement: With no patient present, the antenna system is positioned by the robot scanning mechanism so as to radiate into free-space with no reflective objects within close range. Each antenna element in the linear array is switched on in turn and the reflection coefficient determined for all frequencies via the output voltages from the six-port reflectometer. This represents the complex reflection coefficient of the antenna system and its associated switching network components and is referred to as the ‘empty room’ case. The most significant contribution to the reflection coefficient in this case will be from the antenna apertures.

Second measurement: The procedure outlined above is repeated with a metallic plate placed in close contact with the apertures of each antenna element in the linear array. This is referred to as the ‘flush short circuit’ case. The robot scanner moves the antenna array to a position where a metal plate is automatically in close contact with the aperture plane. The most significant contribution to the reflection coefficient in this case will be from the short circuit plate.

The ‘flush short circuit’ measurement procedure described above is then repeated twice more by placing the metal plate in close contact with the antenna aperture plane but with two waveguide spacers of known length placed, in turn, between the metal plate and the antenna aperture. The two different lengths of waveguide spacer extend the length of the waveguide antenna elements by known amounts and are referred to as ‘offset short circuit calibration standards’.

The three sets of short-circuit data (flush and two offset short circuits) and the empty-room data are used to extract the reflection coefficient of the breast alone from the overall measured reflection coefficient using the antenna array. This is an example of ‘de-embedding’ applied to the measured reflection coefficient data to determine the reflection coefficient of the object in isolation. A description of the de-embedding algorithm used is as follows.

In order to apply the appropriate phase shifts required for synthetic focussing, it is first of all necessary to determine the reflection coefficient at the antenna aperture plane from a knowledge of that determined at the reflectometer reference plane. This requires a knowledge of the scattering parameters of the antenna system which is treated as a ‘black box’ of (linear) components lying between the reflectometer and antenna aperture reference planes.

The reflection coefficients at each plane are related by the following expression:

$$Γ = {S}_{11} + \frac{{S}_{12} {S}_{21} {Γ}_{a}}{( 1 - {S}_{22} {Γ}_{a} )} \qquad ( 3 )$$

where:

Γ=Complex reflection coefficient determined at the reflectometer reference plane.

Γₐ=Complex reflection coefficient determined at the antenna aperture reference plane.

S₁₁,S₂₂,S₁₂,S₂₁ are the elements of the 2×2 antenna system scattering matrix.

Equation (3) can be re-written in the following form:

$$Γ = [ \frac{{S}_{11} - D {Γ}_{a}}{1 - {S}_{22} {Γ}_{a}} ] \qquad ( 4 )$$

where:

D=S₁₁S₂₂-S₁₂S₂₁ is the determinant of the scattering matrix.

Therefore, there are 3 unknown complex coefficients (S₁₁,S₂₂,D) to be determined in (4) to enable the reflection coefficient at the antenna plane to be found from a measurement of the reflection coefficient at the reflectometer reference plane. This requires 3 known calibration standards to be used in the antenna calibration process.

Using one flush and 2 offset short circuits with reflection coefficients of the form Γₐⁿ=−e^{jφ}^{ₙ} (n=1,2,3) leads to the following solution for the antenna calibration coefficients, S₁₁, S₂₂ and D:

$$\begin{matrix}{S}_{22} = ( \frac{{Γ}_{3} {Δ}_{21} - {Γ}_{2} {Δ}_{31} + {Γ}_{1} {Δ}_{32}}{- {Γ}_{3} {Δ}_{21} {ⅇ}^{j {ϕ}_{3}} + {Γ}_{2} {Δ}_{31} {ⅇ}^{j {ϕ}_{2}} - {Γ}_{1} {Δ}_{32} {ⅇ}^{j {ϕ}_{1}}} ) & ( 5 ) \\ D = \frac{{Γ}_{2} ( 1 + {S}_{22} {ⅇ}^{j {ϕ}_{2}} ) - {Γ}_{1} ( 1 + {S}_{22} {ⅇ}^{j {ϕ}_{1}} )}{{Δ}_{21}} & ( 6 ) \\ {S}_{11} = {Γ}_{3} ( 1 + {S}_{22} {ⅇ}^{j {ϕ}_{3}} ) - D {ⅇ}^{j {ϕ}_{3}} & ( 7 )\end{matrix}$$

where in the above:

Γ₁,Γ₂,Γ₃ are the complex reflection coefficients measured at the reflectometer reference plane with calibration standards 1, 2 and 3 fitted to the antenna aperture plane, respectively, and:
Δ₂₁=e^{jφ}^{₂}−e^{jφ}^{₁}
Δ₃₁=e^{jφ}^{₃}−e^{jφ}^{₁}
Δ₃₂=e^{jφ}^{₃}−e^{jφ}^{₂}
φₙ=2βlₙ n=1,2,3

β=Waveguide propagation constant (radians/metre)

lₙ=Length of waveguide offset for the n^{th} calibration standard (metres)

The de-embedded reflection coefficient, Γₐ, is then given by:

$${Γ}_{a} = ( \frac{{S}_{11} - Γ}{D - {S}_{22} Γ} ) \qquad ( 8 )$$

where Γ is the reflection coefficient measured at the reflectometer reference plane.

Equation (8) is evaluated twice—once for the antenna only (‘empty’ case) and once with the patient present. The reflection coefficient of the breast alone referenced to the antenna aperture plane is then found by subtracting the value of Γₐ obtained for the ‘empty’ case from that obtained with patient present. This simple subtraction of the two (complex) reflection coefficients is justified on the basis that multiple reflections between antenna and object are negligible due to the relatively large separation between them (˜10λ at 10 GHz). The imaging algorithm is then applied to the de-embedded reflection coefficient so obtained.

The YIG oscillator 401 of the radar device generates continuous wave (CW) electromagnetic radiation covering a broad frequency bandwidth, i.e. the preferred form imaging system operates in broadband. In the preferred form, the operating frequency band is from 10 GHz to 18 GHz and radiation information is acquired at a number of frequencies throughout the band at each scan location within the synthetic aperture. The broadband frequency domain operation is utilised in order to provide a small focal spot size and hence good image resolution in the down-range direction. In the preferred form radar device, 161 discrete frequencies are used corresponding to a frequency interval of 50 MHz between 10 GHz and 18 GHz. The frequency interval is chosen to be small enough such that aliasing in the down-range direction is avoided in the final 3D radar images for the locations of interest in the image space.

In order to obtain good focusing properties that approach the theoretical diffraction limit of half a wavelength for the size of the focal spot in the transverse plane, the synthetic aperture size needs to be large compared to the wavelength, λ. Therefore, the requirement that D=10λ at the lowest frequency (longest wavelength) follows. If D=30 cm as mentioned previously, then λ=3 cm. Therefore, the minimum frequency of operation for the imaging system is preferably 10 GHz.

The broader the frequency bandwidth, the better the down-range resolution, so as broad a bandwidth as possible is desirable. However, the vast majority of components will only work over a limited band, typically an octave at best. Therefore, 18 GHz is typically the upper frequency of operation given the current performance of available components, giving a bandwidth of 8 GHz.

The frequency interval between steps as the device is swept across the full frequency band is also determined by the need to satisfy the Nyquist sampling criterion. A small enough frequency interval needs to be used so as to avoid grating lobes in the time domain response resulting from an integration over the frequency domain data. This is in turn related to the round-trip time delay from source to receiver via the object under test. The frequency interval is chosen so that alias bands in the time domain response do not lie within the time interval for signals to make a round trip. This time delay can also be represented as an equivalent distance (there and back) in free-space referred to as the Alias-Free Range (AFR). A frequency interval of 50 MHz is used in the preferred form breast imaging system giving 161 frequencies between 10 GHz and 18 GHz.

Denoting the frequency interval by δf, the corresponding separation of alias bands in the time domain, δt, is given by the following equation:

$$δ t = \frac{1}{δ f} \qquad ( 9 )$$

Equation (9) can be used to calculate an equivalent ‘round-trip’ distance in free-space, (AFR), by multiplying δt by 2c where c is the speed of light in free-space to give equation:

$$AFR = 2 c δ t = \frac{2 c}{δ f} \qquad ( 10 )$$

The microwave path length between source and image point and back should be less than the AFR in order to avoid contamination of the radar images from alias responses due to the sampling interval used in the frequency domain. Using δf=50 MHz in equation (10) gives AFR=11.99 meters in free space, which is deemed to be sufficiently large for the proposed imaging system to avoid alias responses. A larger number of frequencies (and therefore a smaller frequency interval) could be used but this has to be offset against the total data acquisition time which must be kept small so as not to inconvenience the patient. For example, the patient should ideally be able to hold there breath for the duration of the scan.

The synthetic aperture method and apparatus described above consisting of an array of small antenna elements that behave collectively like an antenna of the same total physical size but whose characteristics can be reconfigured by manipulation of the relative phase and amplitude weighting applied to each element enables synthetic focusing to an arbitrary point in space via signal processing carried out after the data has been acquired in this piece-wise fashion. This provides a powerful microwave lens that can be focused to an arbitrary location within the breast. This synthetic focusing ability provides the means of imaging small interior features such as malignant tumours. Also, due to the coherent addition of signals obtained from all elements in the synthetic array when focusing to a given point, the signal-to-noise ratio (SNR) of the measurement is improved by a factor N over a single measurement at a single frequency where N is the number of antenna elements in the synthetic array. Furthermore, by making measurements in the frequency domain, one frequency at a time, and then summing up the coherent signals from all antenna elements at all frequencies (to get a time domain response) the signal to noise ratio is further enhanced by a factor F where F is the number of discrete frequencies used.

By coherent addition of signals at the designated synthetic focal point, the imaging device becomes very sensitive to scattered fields located at the focus. The coherent addition is carried out over all antenna locations and at all frequencies. A useful figure of merit is the increase in sensitivity of the imaging device as a result of focusing signals in this way and this is equal to the product of the number of antenna elements with the number of frequencies. This is also equal to the improvement in signal-to-noise ratio over and above a measurement of reflectivity carried out by a single antenna at a single frequency. For the breast imaging device this factor is 161×1024=164,864, which is equivalent to an improvement of about +52 dB. This is more than sufficient to overcome the two-way attenuation of signals in the breast tissue and skin which, at a depth of 5 cm at a frequency of 18 GHz, is about −40 dB. To this end, higher frequencies than 18 GHz could be contemplated with a subsequent improvement in resolution in transverse and down-range directions.

In the preferred form imaging system, frequencies in the range 10 GHz to 18 GHz are used. In general, attenuation in the breast tissue increases with increasing frequency. The benefit of using higher frequencies is the improved spatial resolution due to the reduced wavelength. The attenuation encountered does not pose difficulties for the preferred form imaging system due to the enhancement in sensitivity (e.g. +52 dB) obtained as a result of coherent addition of received signals over a large number of antenna elements (e.g. 1024) along with integration over (e.g. 161) frequencies. Thus, the imaging system can accommodate higher microwave frequencies, which enhances the resolution compared to lower-frequency systems.

Also, the nature of electromagnetic scattering from small objects compared to the wavelength, such as the small malignant tumours of interest in breast cancer screening, needs to be considered. Such objects reflect incident energy back to the receiving antenna according to Rayleigh scattering theory. In Rayleigh scattering, the back-scattered power is proportional to the fourth power of the frequency. Therefore, the back-scattered signal from a small embedded object in the breast is 1.8⁴ times larger at 18 GHz than it is at 10 GHz. This is a factor of approximately 10.5 or +10.2 dB. This enhanced scattering at the high-frequency end of the proposed frequency spectrum also helps to offset the increased attenuation in the breast tissue at the higher frequencies.

In the preferred form, the imaging system is non-contact and does not require a liquid immersion medium surrounding the breast and antenna system. In addition, the separation between antennas and the breast is typically of the order of ten wavelengths at the lowest frequency of operation (about 30 cm at 10 GHz). This is advantageous over some prior microwave systems that utilise both a liquid coupling medium and have antenna elements either in contact with the breast or in close proximity to it. The motivation for including a liquid medium around the breast is one of impedance matching with respect to the properties of the interior breast tissue. Reflections from the skin layer can be large thereby reducing the amount of energy entering the breast. If the dielectric constant of the liquid medium is similar to that of breast tissue then the amount of microwave energy penetrating the breast is maximised. The only residual effects that remain are reflections from the skin and attenuation in all media.

It will be appreciated that alternative forms of the imaging system may have antenna element(s) that directly contact the patient's breast or that are coupled to the patient's breasts via a liquid immersion medium, matching layer or matching plate having the appropriate dielectric constant. With a direct contact imaging system, a robot or other scanning mechanism would be arranged to sequentially move the array of antenna elements directly into contact with the breast at each of the predetermined scan locations. Similarly, with a liquid, layer or plate coupled system a robot or other scanning mechanism would be arranged to sequentially move the array of antenna elements relative to the breast to obtain the radiation information at each of the predetermined scan locations defining the synthetic or real aperture relative to the breast. It will be appreciated that there are various coupling configurations possible. For example, the liquid immersion medium could be applied directly to the patient's breasts or alternatively to the antenna elements. Similarly, the matching layer or plate could be fixed relative to the patient's breasts or alternatively fixed relative to the antenna elements.

The preferred form imaging system has been described as operating in the range of 10 GHz-18 GHz, but the system could be arranged to operate within other higher or lower frequency ranges in the microwave band. For example, the imaging system could employ frequencies below 10 GHz or above 18 GHz. An example of one possible higher frequency band is 20 GHz-40 GHz. The frequency range employed will ultimately depend on the capabilities of the componentry. Further, the number of discrete frequencies utilised within the selected frequency range can be adjusted to suit design requirements. Preferably the imaging system utilises at least 10 discrete frequencies, more preferably at least 100 discrete frequencies, and even more preferably at least 161 discrete frequencies. Ultimately, the number of discrete frequencies utilised must be sufficient to enable the generation of a reasonable 3D radar image and will depend on other design parameters such as frequency range, Nyquist sampling criterion, AFR, amount of radiation data required etc.

It will be appreciated that the aperture size within which radiation information is obtained can be altered as desired. Further, the number of predetermined measuring locations within the aperture and their respective spacings may be adjusted for specific requirements. For example, the number of predetermined measuring locations within the aperture may be increased to provide more radiation information in order to enhance the quality of the 3D radar image generated.

The synthetic focusing algorithm has been described in the context of a breast imaging system, but it will be appreciated that the algorithm may be adapted to generate 3D radar images of other body parts and their internals. For example, the imaging system could be arranged to scan any other body part and use the synthetic focusing algorithm to generate 3D radar images that depict bone, brain, skin, muscle, collagen, ligaments, tendons, cartilige, organs, or the lymphatic system or any other part of the body. In particular, the imaging system may be utilised to scan other body parts to obtain radiation information and external surface profile information, and then employ the synthetic focusing algorithm to generate a 3D radar image of the body part by focusing the radiation information within the body part. For example, the imaging system may be able to generate a 3D radar image of a limb, such as a leg or arm, by scanning to obtain radiation information and skin/external surface profile information about the leg or arm, and then synthetically focusing the radiation information using the algorithm to generate the 3D radar image. The 3D radar image of the leg or arm could then be utilised to assess the skin, bone, joints, tendons, muscle, ligaments or other soft tissues of the leg or arm. A similar process may be utilised to generate 3D radar images of the head, chest, or torso to assess the brain and other organs, bones and tissues. The 3D radar images generated could be utilised for various diagnostic purposes. For example, the images could be utilised to detect bone fractures, internal bleeding, or brain tumours. Further, the imaging system may be utilised to image animal body parts.

It will be appreciated that the synthetic foucussing method can be arranged to generate complete 3D radar images of body parts or partial 3D radar images of particular areas within the body parts. In particular, the synthetic focusing method utilises the skin surface profile information to focus the radiation information within the body part to generate the partial or complete 3D radar images. For breast imaging, knowledge or estimates of the skin thickness, skin dielectric constant, and breast tissue dielectric constant, along with the external surface profile information, enable the radiation information to be synthetically focused within the breast. Similarly, to image other body parts, knowledge or estimates of the skin thickness, skin dielectric constant and the thickness and dielectric constants of the various other dielectric interfaces (for example muscle, soft tissue, organs, bone etc) within the body part may be utilised with the surface profile information to synthetically focus the radiation information within the body part to generate the desired 3D radar images. For example, for brain imaging, knowledge or estimates of the thickness of the skin and skull, and the dielectric constants of the skin, skull and brain, along with surface profile information of the head, enable the synthetic focusing algorithm to focus radiation information (radar data) to within the head to generate a 3D radar image of the brain. Therefore, the imaging system may scan a body part to obtain radation information and then employ the synthetic focusing algorithm to focus that radaition information using surface profile information and knowledge or estimates of the properties (thickness and dielectric constants for example) of the various dielectric interfaces within the body part to generate the required 3D radar images.

Experimental Results—Pre-Clinical Trial

A prototype imaging system for breast cancer screening that utilises the synthetic focusing algorithm has been constructed and trialed on patients. The prototype was constructed substantially according to the preferred design specifications discussed above. In particular, the prototype was arranged to obtain radar reflectivity data (radiation information) over a synthetic aperture approximately 27 cm×27 cm in 0.85 cm steps giving a data array 32 elements by 32 elements. Further, the prototype was arranged to obtain the radar reflectivity measurements (phase and amplitude) at 50 MHz increments in the frequency band of 10 GHz-18 GHz for each of the 1024 synthetic aperture scan locations. During the scan, the patients lay on their backs with their breasts exposed and the antenna aperture plane was located approximately 30 cm above the patients. The prototype utilised a 3D laser profiler to scan the patient's breast giving geometrical information of the breast's outer profile. This information was combined with the radar data to generate a three-dimensional radar image of the breast interior. An estimate of the skin thickness and dielectric properties of the skin and normal breast tissue were utilised to generate a focused interior image. A skin thickness of 2 mm was assumed with a skin tissue dielectric constant of 40. Normal breast tissue was assumed to have a dielectric constant of 9.

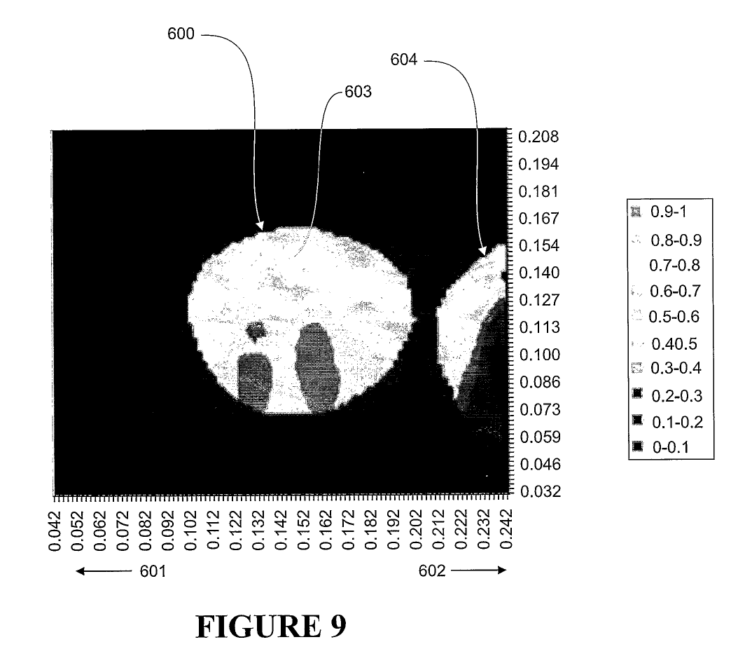

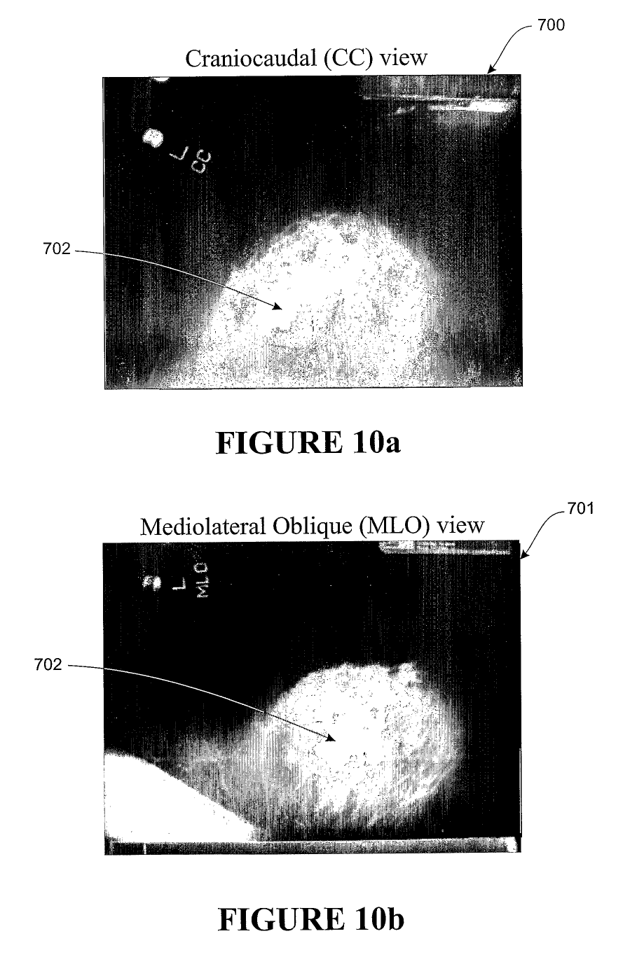

By way of example, the results for one patient of the pre-clinical trial will be explained with reference to FIGS. 9, 10a and 10b. FIG. 9 shows a single two-dimensional slice 600 of the resulting three-dimensional radar image of the breast interior for one of the patients. This slice 600 is evaluated at a depth of 12 mm below the breast surface (arrow 601 is toward the patient's head and arrow 602 is toward the patient's feet). A suspected tumour 603 appears as a distinct oval feature with a radar intensity higher than that of the surrounding tissue. The external rib cage 604 is also visible in the slice 600. The three-dimensional radar image captured was compared to the corresponding mammogram images of the same patient shown in FIGS. 10a and 10b (craniocaudal 700 and mediolateral oblique 701 views). The mammograms 700, 701 clearly show a large suspected tumour 702 (˜2 cm in diameter) located in the upper outer quadrant of the breast. Whilst no direct comparison between mammograms and radar images is possible (since, unlike radar images, mammograms involve breast compression) the radar image captured clearly identified the presence of a large suspected tumour located in the correct part of the breast. In particular, the radar images captured by the imaging system showed a suspected tumour, the location and size of which was consistent with the suspected tumour shown in the mammogram images of FIGS. 10a and 10b.

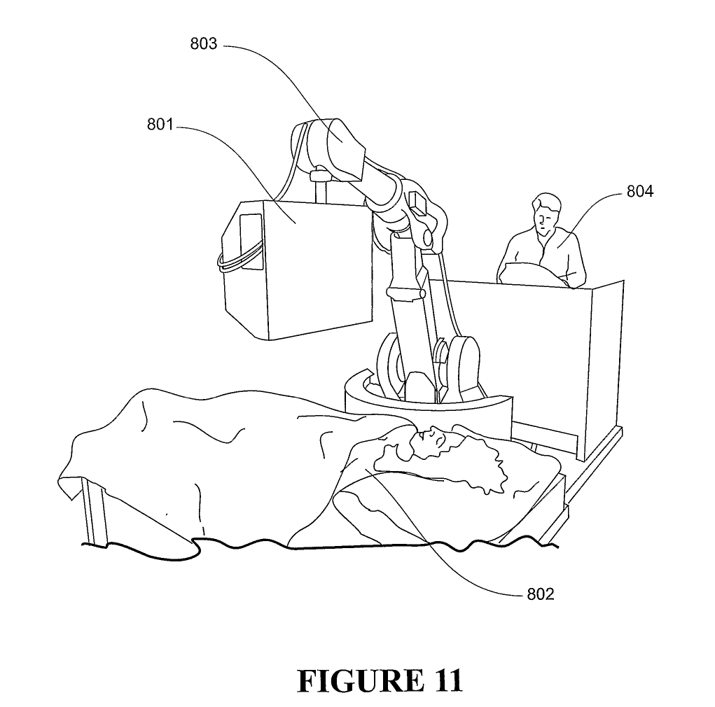

FIG. 11 shows the prototype imaging system used in the pre-clinical trial. The sensor head 801 is moved relative to the patient 802 by a robot scanning mechanism 803 as previously described. An operator 804 controls the imaging system via a control system. During the scan, the patient's breasts are exposed and the radar device and 3D profiler of the sensor head 801 are operated to obtain the radiation and surface profile information so that 3D radar images of the breasts can be generated.

The foregoing description of the invention includes preferred forms thereof. Modifications may be made thereto without departing from the scope of the invention as defined by the accompanying claims.

1. A method of generating a three-dimensional radar image of a body part having multiple image points comprising:
receiving radiation information obtained at an array of scan locations relative to the body part, the radiation information being obtained at multiple microwave frequencies at each of the scan locations;
receiving surface profile information relating to the body part;
receiving estimates of body part properties;
constructing each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and
generating the 3D radar image of the body part based on the values of each of the image points.

2. A method according to claim 1 wherein the body part properties comprise: estimates of the thickness and dielectric constant of dielectric interfaces of the body part between the scan locations and the image point; and estimates of the dielectric constant of the body part in the vicinity of the image point.

3. A method according to claim 1 wherein the body part properties comprise: estimates of the thickness and dielectric constant of the skin dielectric interface; and the dielectric constant of the body part in the vicinity of the image point.

4. A method according to claim 3 wherein the body part is a human breast and the body part properties comprise: estimates of the thickness and dielectric constant of the skin dielectric interface of the breast; and the dielectric constant of the breast tissue.

5. A method according to claim 1 wherein determining the minimum optical paths between each scan location and the image point being constructed comprises: mapping the valid optical paths between each scan location and the image point using Snell's Law of Refraction and selecting the minimum optical path from the valid optical paths.

6. A method according to claim 1 wherein the values of the image points are radar intensity values.

7. A method according to claim 1 further comprising displaying the three-dimensional radar image of the body part.

8. A method according to claim 1 wherein the radiation information is obtained at each scan location at multiple discrete frequencies of at least 10 GHz.

9. A method according to claim 8 wherein the radiation information is obtained at multiple discrete frequencies in the frequency range of approximately 10 GHz-18 GHz.

10. A method according to claim 8 wherein the radiation information is obtained at at least 10 discrete frequencies.

11. A method according to claim 8 wherein the radiation information is obtained at at least 100 scan locations relative to the body part.

12. A system for generating a three-dimensional radar image of a body part having multiple image points comprising:
an input for receiving input data comprising: radiation information obtained at an array of scan locations relative to the body part, the radiation information being obtained at multiple microwave frequencies at each of the scan locations; surface profile information relating to the body part; and estimates of body part properties;
a processor arranged to process the input data to construct each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and
an output for sending output data relating to the image point values for the generation of the 3D radar image of the body part.

13. A system according to claim 12 wherein the body part properties comprise: estimates of the thickness and dielectric constant of dielectric interfaces of the body part between the scan locations and the image point; and estimates of the dielectric constant of the body part in the vicinity of the image point.

14. A system according to claim 12 wherein the body part properties comprise: estimates of the thickness and dielectric constant of the skin dielectric interface; and the dielectric constant of the body part in the vicinity of the image point.

15. A system according to claim 14 wherein the body part is a human breast and the body part properties comprise: estimates of the thickness and dielectric constant of the skin dielectric interface of the breast; and the dielectric constant of the breast tissue.

16. A system according to claim 12 wherein the processor is arranged to determine the minimum optical paths between each scan location and the image point being constructed by mapping the valid optical paths between each scan location and the image point using Snell's Law of Refraction and selecting the minimum optical path from the valid optical paths.

17. A system according to claim 12 wherein the values of the image points are radar intensity values.

18. A system according to claim 12 further comprising an output display for receiving the output data and displaying the three-dimensional radar image of the body part.

19. A system according to claim 12 wherein the radiation information is obtained at each scan location at multiple discrete frequencies of at least 10 GHz.

20. A system according to claim 19 wherein the radiation information is obtained at multiple discrete frequencies in the frequency range of approximately 10 GHz-18 GHz.

21. A system according to claim 19 wherein the radiation information is obtained at at least 10 discrete frequencies.

22. A system according to claim 19 wherein the radiation information is obtained at at least 100 scan locations relative to the body part.

23. A computer program for generating a three-dimensional radar image of a body part having multiple image points, the program being arranged to:
receive input data comprising: radiation information obtained at an array of scan locations relative to the body part, the radiation information being obtained at multiple microwave frequencies at each of the scan locations; surface profile information relating to the body part; and estimates of body part properties;
process the input data to construct each image point by: determining the minimum optical paths between each scan location and the image point based on the scan locations, surface profile information and body part properties; phase-shifting the radiation information based on the minimum optical paths to equalise the radiation information; and then summing the equalised radiation information over all scan locations and all frequencies to provide a value for the image point; and
output data relating to the image point values for the generation of the 3D radar image of the body part.

24. A computer program according to claim 23 wherein the body part properties comprise: estimates of the thickness and dielectric constant of dielectric interfaces of the body part between the scan locations and the image point; and estimates of the dielectric constant of the body part in the vicinity of the image point.

25. A computer program according to claim 23 wherein the body part properties comprise: estimates of the thickness and dielectric constant of the skin dielectric interface; and the dielectric constant of the body part in the vicinity of the image point.

26. A computer program according to claim 25 wherein the body part is a human breast and the body part properties comprise: estimates of the thickness and dielectric constant of the skin dielectric interface of the breast; and the dielectric constant of the breast tissue.

27. A computer program according to claim 23 wherein the computer program is arranged to determine the minimum optical paths between each scan location and the image point being constructed by mapping the valid optical paths between each scan location and the image point using Snell's Law of Refraction and selecting the minimum optical path from the valid optical paths.

28. A computer program according to claim 23 wherein the values of the image points are radar intensity values.

29. A computer program according to claim 23 wherein the computer program outputs data to an output display for displaying the three-dimensional radar image of the body part.

30. A computer program according to claim 23 wherein the radiation information is obtained at each scan location at multiple discrete frequencies of at least 10 GHz.

31. A computer program according to claim 30 wherein the radiation information is obtained at multiple discrete frequencies in the frequency range of approximately 10 GHz-18 GHz.

32. A computer program according to claim 30 wherein the radiation information is obtained at at least 10 discrete frequencies.

33. A computer program according to claim 30 wherein the radiation information is obtained at at least 100 scan locations relative to the body part.
